(19) United States
(12) Patent Application Publication
(10) Pub. No.: US 2011/0110891 A1
(43) Pub. Date: May 12, 2011

(54) Methods of Treating Hepatitis C Virus Infection

(76) Inventors: Melanie Ott, San Francisco, CA (US); Eva Herker, San Francisco, CA (US)

(21) Appl. No.: 12/991,380

(22) PCT Filed: May 11, 2009
(86) PCT No.: PCT/US09/02900

§ 371 Date: Jan. 24, 2011

Related U.S. Application Data
(60) Provisional application No. 61/052,008, filed on May 9, 2008.

(51) Int. Cl. A61K 38/21; A61K 31/7056; A61P 31/14; A61K 31/7076; A61K 31/662; A61K 31/404; A61K 31/4412; A61K 31/352; A61K 31/5377; A61K 31/5025

(52) U.S. Cl. 424/85.7; 514/43; 514/46; 514/119; 514/418; 514/350; 514/456; 514/235.2; 514/248

(57) ABSTRACT

The present disclosure provides methods of treating a hepatitis C virus infection. The methods generally involve administering to an individual in need thereof an effective amount of an active agent that inhibits a RAS-RAF-MEK-ERK signal transduction pathway.

### CROSS-REFERENCE

This application claims the benefit of U.S. Provisional Patent Application No. 61/052,008, filed May 9, 2008, which application is incorporated herein by reference in its entirety.

### STATEMENT REGARDING FEDERALLY SPONSORED RESEARCH

The U.S. government has certain rights in this invention, pursuant to grant no. 1R03AI069090-01A1 awarded by the National Institutes of Allergy and Infectious Diseases.

### BACKGROUND

Among the key signaling pathways regulating mammalian cell growth and differentiation is the ERK (extracellular signal-regulated kinase) pathway. ERK is a member of the mitogen-activated protein kinases (MAPK) family of protein kinases. The activation of ERK requires a cascade mechanism whereby ERK is phosphorylated by an upstream kinase MAPKK (MEK) which is in turn phosphorylated by a third kinase MAPKKK (MEKK) also known as RAF. ERK has two closely related isoforms of 44 kDa and 42 kDa, corresponding to ERK-1 and ERK-2 respectively.

Enhancement of MEK or ERK activity in response to cell stimulation involves phosphorylation at residues located within the activation lip of each kinase. In the case of MEK, phosphorylation at two serine residues (Ser²¹⁸/Ser²²² in human MEK-1; Ser²²²/Ser²²⁶ in human MEK-2) by upstream protein kinase RAF-1, leads to maximal enzyme activation. MEK 1/2 subsequently activates ERK 1/2 by phosphorylating regulatory threonine and tyrosine residues (Th²⁰²/Tyr²⁰⁴ in human ERK-1; Thr¹⁸⁵/Tyr¹⁸⁷ in human ERK-2.

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Currently, treatments for HCV infection include weekly injections of pegylated interferon alfa (IFN-α) combined with twice-daily oral doses of ribavirin. Nevertheless, even with combination therapy using pegylated IFN-α plus ribavirin, 40% to 50% of patients fail therapy, i.e., 40% to 50% of patients are nonresponders or relapsers. These patients currently have no effective therapeutic alternative. Patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

There is a need in the art for therapies for HCV infection.

### LITERATURE

Schmitz et al. (2008) J. Hepatol. 48:83-90; Zhao et al. (2007) Cell Prolif. 40:508; Andersson et al. (2006) J. Cell Sci. 119:2246; U.S. Patent Publication No. 2005/0215627; PCT Publication No. WO 00/40237; Bürckstümmer et al. (2006) FEBS Lett. 580:575; Murata et al. (2005) Virol. 340:105; U.S. Patent Publication No. 2008/0176846.

### SUMMARY OF THE INVENTION

The present disclosure provides methods of treating a hepatitis C virus infection. The methods generally involve administering to an individual in need thereof an effective amount of an active agent that inhibits a RAS-RAF-MEK-ERK signal transduction pathway.

### BRIEF DESCRIPTION OF THE DRAWINGS

FIGS. 1A-D provide exemplary amino acid sequences of Ras polypeptides.

FIGS. 2A-C provide exemplary amino acid sequences of Raf polypeptides.

FIGS. 3A and 3B provide exemplary amino acid sequences of MEK amino acid sequences.

FIGS. 4A and 4B provide exemplary amino acid sequences of ERK amino acid sequences.

FIG. 5 depicts the effect of RAS-RAF-MEK-ERK pathway inhibitors on level of HCV RNA in supernatants of infected cell cultures.

FIG. 6 depicts the effect of RAS-RAF-MEK-ERK pathway inhibitors on levels of intracellular HCV RNA.

FIG. 7 depicts the effect of RAS-RAF-MEK-ERK pathway inhibitors on production of infectious HCV.

FIG. 8 depicts the effect of RAS-RAF-MEK-ERK pathway inhibitors on production of infectious virus.

FIG. 9 depicts the effect of HCV core on ERK2 phosphorylation.

FIG. 10 depicts the effect of MEK inhibition on viral entry.

FIG. 11 depicts the effect of MKE1/2 inhibition on viral assembly.

FIG. 12 depicts the effect of MEK1/2 inhibition on HCV RNA replication.

FIG. 13 depicts the effect of inhibitors on HCV RNA release.

### DEFINITIONS

As used herein, the term “Flaviviridae virus” includes any member of the family Flaviviridae, including, but not limited to, Dengue virus, including Dengue virus 1, Dengue virus 2, Dengue virus 3, Dengue virus 4 (see, e.g., GenBank Accession Nos. M23027, M19197, A34774, and M14931); Yellow Fever Virus; West Nile Virus; Japanese Encephalitis Virus; St. Louis Encephalitis Virus; Bovine Viral Diarrhea Virus (BVDV); and Hepatitis C Virus (HCV); and any serotype, strain, genotype, subtype, quasispecies, or isolate of any of the foregoing.

By “HCV” herein is meant any one of a number of different genotypes and isolates of hepatitis C virus. Representative HCV genotypes and isolates include: H77, the “Chiron” isolate, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type 1d, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6 and type 6a.

The terms “polypeptide” and “protein” are used interchangeably throughout the application to refer to at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. Normally, the amino acids are in the (S) or L-configuration. If non-naturally occurring side chains are used, non-amino acid substituents may be used, for example to prevent or retard in vivo degradation. Naturally occurring amino acids are normally used and the protein is a cellular protein that is either endogenous or expressed recombinantly.

A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes, but is not limited to, the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

As used herein, “subject” or “individual” or “patient” refers to any subject for whom or which therapy is desired, and generally refers to the recipient of the therapy to be practiced according to the invention. The subject can be any vertebrate, but will typically be a mammal. If a mammal, the subject will in many embodiments be a human, but may also be a domestic livestock, laboratory subject or pet animal.

The terms “treat,” “treating,” “treatment” and the like are used interchangeably herein and mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed the disease such as enhancing the effect of a viral infection. “Treating” as used herein covers treating a disease in a vertebrate and particularly a mammal and most particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

The term “prodrug,” as used herein, refers to a derivative of a drug molecule that requires a chemical or enzymatic biotransformation in order to release the active parent drug in the body.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

The term “effective amount” or “therapeutically effective amount” means a dosage sufficient to provide for treatment for the disease state being treated or to otherwise provide the desired effect (e.g., reduction of viral load). The precise dosage will vary according to a variety of factors such as subject-dependent variables (e.g., age, immune system health, etc.), the disease (e.g., the particular viral strain), and the treatment being effected. In the case of treatment of HCV infection, an “effective amount” can be considered that amount sufficient to reduce the HCV viral load in a subject, as described in more detail below.

As used herein, “pharmaceutically acceptable derivatives” of an active compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

As used herein, “pharmaceutically acceptable carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed., Mack Publishing Col, Easton Pa. 18042, USA). Pharmaceutically acceptable excipients have been amply described in a variety of publications, including, for example, A. Gennaro (2000) “Remington: The Science and Practice of Pharmacy,” 20th edition, Lippincott, Williams, & Wilkins; Remington's Pharmaceutical Sciences, 14th Ed. or latest edition, Mack Publishing Col, Easton Pa. 18042, USA; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an active agent” includes a plurality of such agents and reference to “the HCV genotype” includes reference to one or more HCV genotypes and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of treating an infection by a virus that is a member of the family Flaviviridae, e.g., hepatitis C virus (HCV). The methods generally involve administering to an individual in need thereof an effective amount of an inhibitor of a RAS-RAF-MEK-ERK signal transduction cascade. The present disclosure provides methods of treating liver fibrosis, generally involving administering to an individual in need thereof an effective amount of an inhibitor of a RAS-RAF-MEK-ERK signal transduction cascade. The present disclosure also provides methods of treating steatosis.

### Treating HCV Infection

The present disclosure provides methods of treating an HCV infection, the methods generally involving administering to an individual in need thereof (e.g., an HCV-infected individual) an effective amount of an agent that inhibits a RAS-RAF-MEK-ERK signal transduction cascade.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

Whether a subject method is effective in treating an HCV infection can be determined by assessing the effect of an inhibitor of the RAS-RAF-MEK-ERK signal transduction cascade in a humanized mouse model suitable for HCV infection, e.g., the human liver-uPA-SCID mouse model. Meuleman et al. (2005) Hepatol. 41:847; Meuleman and Leroux-Roels (2008) Antiviral Res. 80:231.

In some embodiments, an effective amount of an agent that inhibits a RAS-RAF-MEK-ERK signal transduction cascade is an amount that reduces cell-to-cell spread of HCV. For example, in some embodiments, an effective amount of an agent that inhibits a RAS-RAF-MEK-ERK signal transduction cascade is an amount that reduces spread of HCV from one cell to another by at least about 10%, at least about 20%, at least about 25%, at least about 30%, at least about 40%, at least about 50%, at least about 60%, at least about 70%, at least about 80%, or more than 80%, compared to the cell-to-cell spread in the absence of the agent. In other words, in some embodiments, an effective amount of an agent that inhibits a RAS-RAF-MEK-ERK signal transduction cascade can be considered an amount that reduces the rate of spread of HCV from an HCV-infected cell to an uninfected cell by at least about 10%, at least about 20%, at least about 25%, at least about 30%, at least about 40%, at least about 50%, at least about 60%, at least about 70%, at least about 80%, or more than 80%, compared to the rate of spread in the absence of the agent. For example, over a given period of time, an effective amount of an agent that inhibits a RAS-RAF-MEK-ERK signal transduction cascade can reduce the number of uninfected cells in an individual that become infected from HCV produced by an HCV-infected cell in the same individual, by at least about 10%, at least about 20%, at least about 25%, at least about 30%, at least about 40%, at least about 50%, at least about 60%, at least about 70%, at least about 80%, or more than 80%, compared to the number of uninfected cells that become infected over the same time period in the absence of the agent.

In some embodiments, an effective amount of an agent that inhibits a RAS-RAF-MEK-ERK signal transduction cascade is an amount that, when contacted with an HCV-infected cell, reduces the number of infectious HCV particles produced by the HCV-infected cell by at least about 10%, at least about 20%, at least about 25%, at least about 30%, at least about 40%, at least about 50%, at least about 60%, at least about 70%, at least about 80%, or more than 80%, compared to the number of infectious HCV particles produced by the HCV-infected cell in the absence of the agent (e.g., compared to the number of infectious HCV particles produced by the HCV-infected cell not contacted with the agent).

In some embodiments, an effective amount of an agent that inhibits a RAS-RAF-MEK-ERK signal transduction cascade is an amount that, when administered to an HCV-infected individual in one or more doses, alone (e.g., in monotherapy) or in combination with one or more additional anti-viral agents, is effective to reduce a serum level of HCV in the individual. For example, in some embodiments, an effective amount of an agent that inhibits a RAS-RAF-MEK-ERK signal transduction cascade is an amount that, when administered to an HCV-infected individual in one or more doses, alone or in combination with one or more additional anti-viral agents, is effective to reduce the level of serum HCV in the individual to from about 1000 genome copies/mL serum to about 5000 genome copies/mL serum, to from about 500 genome copies/mL serum to about 1000 genome copies/mL serum, or to from about 100 genome copies/mL serum to about 500 genome copies/mL serum. In some embodiments, an effective amount of an agent that inhibits a RAS-RAF-MEK-ERK signal transduction cascade is an amount that, when administered to an HCV-infected individual in one or more doses, alone or in combination with one or more additional anti-viral agents, is effective to reduce HCV viral load to lower than 100 genome copies/mL serum.

In some embodiments, an effective amount of an agent that inhibits a RAS-RAF-MEK-ERK signal transduction cascade is an amount that, when administered to an HCV-infected individual in one or more doses, alone or in combination with one or more additional anti-viral agents, is effective to achieve a 1.5-log, a 2-log, a 2.5-log, a 3-log, a 3.5-log, a 4-log, a 4.5-log, or a 5-log reduction in viral titer in the serum of the individual.

In some embodiments, an effective amount of an agent that inhibits a RAS-RAF-MEK-ERK signal transduction cascade is an amount that, when administered to an HCV-infected individual in one or more doses, alone or in combination with one or more additional anti-viral agents, is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

### Measuring HCV Viral Load

Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT), developed by Gen-Probe Inc. (San Diego) and Chiron Corporation; and sold by Chiron Corporation under the trade name Procleix®, which NAT simultaneously tests for the presence of HIV-1 and HCV. See, e.g., Vargo et al. (2002) Transfusion 42:876-885.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of an agent that inhibits a RAS-RAF-MEK-ERK signal transduction cascade is an amount that, when administered to an HCV-infected individual in one or more doses, alone or in combination with one or more additional anti-viral agents, is effective to reduce ALT levels to less than about 45 IU/ml serum.

In some embodiments, an effective amount of an agent that inhibits a RAS-RAF-MEK-ERK signal transduction cascade is an amount that, when administered to an HCV-infected individual in one or more doses, alone or in combination with one or more additional anti-viral agents, is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

### Liver Fibrosis

The present disclosure provides methods of treating liver fibrosis (including forms of liver fibrosis resulting from, or associated with, HCV infection), generally involving administering to an individual in need thereof an effective amount of an agent that inhibits a RAS-RAF-MEK-ERK signal transduction cascade.

Whether a subject method for treating liver fibrosis is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that effects a change of one unit or more in the fibrosis stage based on pre- and post-therapy liver biopsies. In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that reduces liver fibrosis by at least one unit in the METAVIR, the Knodell, the Scheuer, the Ludwig, or the Ishak scoring system.

In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include .alpha.-2-macroglobulin, haptoglobin, gamma globulin, apolipoprotein A, and gamma glutamyl transpeptidase.

In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether a subject method is effective in reducing the incidence of a disorder associated with cirrhosis of the liver can readily be determined by those skilled in the art.

Reduction in liver fibrosis increases liver function. In some embodiments, a subject method provides for an increase in liver function. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splancnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splancnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that is effective to increase liver function by at least about 10%, at least about 20%, at least about 30%, at least about 40%, at least about 50%, at least about 60%, at least about 70%, at least about 80%, or more. In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that is effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. In some embodiments, an effective amount of an agent that inhibits a RAS-RAF-MEK-ERK signal transduction cascade is an amount that, when administered to an individual in one or more doses, alone or in combination with one or more additional therapeutic agents, is an amount that is effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Hepatic Steatosis

The present disclosure provides methods of treating hepatic steatosis in an individual, generally involving administering to an individual in need thereof an effective amount of an agent that inhibits a RAS-RAF-MEK-ERK signal transduction cascade.

In some embodiments, an effective amount of an agent that inhibits a RAS-RAF-MEK-ERK signal transduction cascade is an amount that, when administered in one or more doses, is effective to reduce the amount of fat in the liver (by weight) by at least about 5%, at least about 10%, at least about 15%, at least about 20%, at least about 25%, or more, compared to the amount of fat in the liver in an individual not treated with the agent.

Whether the extent of steatosis has been reduced by treatment with an agent that inhibits a RAS-RAF-MEK-ERK signal transduction cascade can be determined using standard methods, e.g., liver biopsy followed by analysis of the biopsied tissue for fat levels. When the amount of fat in the liver is greater than 5-10% by weight, a diagnosis of hepatic steatosis is made. A reduction of the amount of fat in the liver is an indication of efficacy of treatment with an agent that inhibits a RAS-RAF-MEK-ERK signal transduction cascade.

### RAS-RAF-MEK-ERK Pathway Members

As used herein, the term “RAS-RAF-MEK-ERK pathway member” refers to one or more of the following: a “RAS polypeptide,” a “RAF polypeptide,” a “MEK polypeptide,” and an “ERK polypeptide.” The terms “RAS-RAF-MEK-ERK cascade” and “RAS-RAF-MEK-ERK pathway” are used interchangeably herein. Structure-function relationships of the members of a RAS-RAF-MEK-ERK pathway are known in the art and have been described amply in the literature. See, e.g., Kolch (2000) Biochem. J. 351:289. An agent that inhibits a RAS-RAF-MEK-ERK signal transduction cascade includes, but is not limited to, an agent that inhibits a RAS polypeptide, e.g., inhibits enzymatic activity of a RAS polypeptide; an agent that inhibits RAF, e.g., inhibits an enzymatic activity of a RAF polypeptide; an agent that inhibits MEK, e.g., inhibits an enzymatic activity of a MEK polypeptide; and an agent that inhibits ERK, e.g., inhibits an enzymatic activity of an ERK polypeptide, e.g., an ERK-1 polypeptide or an ERK-2 polypeptide. Two or more of such agents can be used in a subject method.

RAS: As used herein, the term “RAS polypeptide” refers to a polypeptide encoded by a member of the ras oncogene family. This gene family includes N-ras (neuroblastoma cell line), H-ras (Harvey murine sarcoma virus), and the alternatively spliced K-ras (Kirsten murine sarcoma virus). A Ras polypeptide has a molecular weight of about 21 kDa. Ras polypeptides exhibit GTPase activity, e.g., a RAS polypeptide binds to and hydrolyzes GTP. Active RAS (e.g., GTP-bound RAS) binds to a Raf kinase with high affinity, and effect translocation of the Raf kinase to the cell membrane.

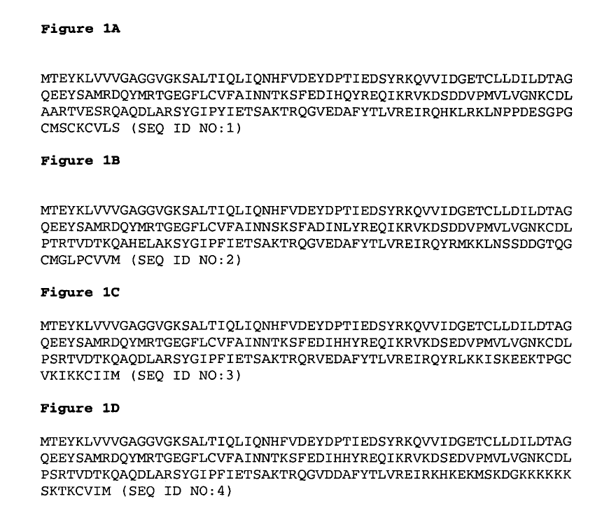

The term “RAS polypeptide” includes a polypeptide that comprises an amino acid sequence having at least about 75%, at least about 80%, at least 85%, at least about 90%, at least about 95%, at least about 98%, at least about 99% or 100% sequence identity to the amino acid sequence set forth in FIG. 1A (H-ras). The term “RAS polypeptide” includes a polypeptide that comprises an amino acid sequence having at least about 75%, at least about 80%, at least 85%, at least about 90%, at least about 95%, at least about 98%, at least about 99% or 100% sequence identity to the amino acid sequence set forth in FIG. 1B (N-ras).

The term “RAS polypeptide” includes a polypeptide that comprises an amino acid sequence having at least about 75%, at least about 80%, at least 85%, at least about 90%, at least about 95%, at least about 98%, at least about 99% or 100% sequence identity to the amino acid sequence set forth in FIG. 1C (K-ras, isoform 2A).

The term “RAS polypeptide” includes a polypeptide that comprises an amino acid sequence having at least about 75%, at least about 80%, at least 85%, at least about 90%, at least about 95%, at least about 98%, at least about 99% or 100% sequence identity to the amino acid sequence set forth in FIG. 1D (K-ras, isoform 2B).

RAF: As used herein, the term “RAF polypeptide” refers to a MAPKKK (MAPK kinase kinase, also referred to as MAP3K) polypeptide having Ser/Thr protein kinase activity and having a molecular weight in a range of about 70 kDa to about 100 kDa. At least three mammalian RAF proteins have been identified, RAF-1, A-RAF and B-RAF. Phosphorylated RAF activates MEK1 and MEK2 by phosphorylation of two serine residues at positions 217 and 221 in the activation loop.

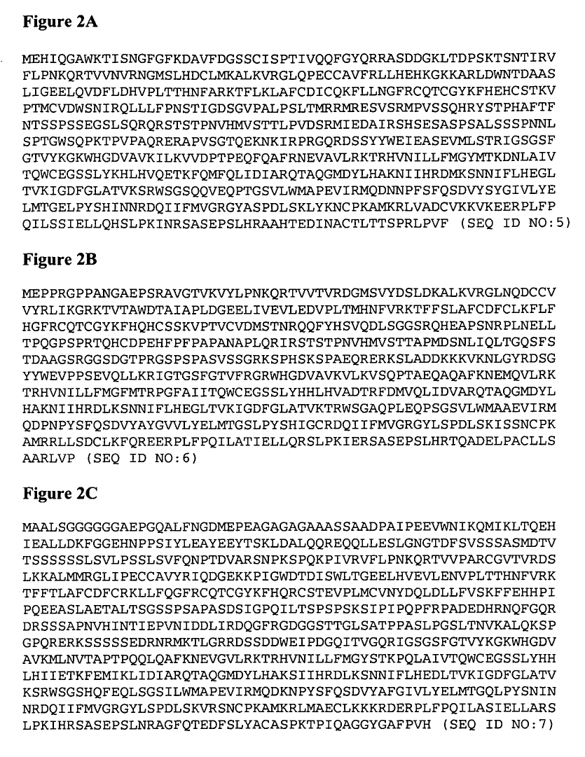

The term “RAF polypeptide” includes a polypeptide that comprises an amino acid sequence having at least about 75%, at least about 80%, at least 85%, at least about 90%, at least about 95%, at least about 98%, at least about 99% or 100% sequence identity to the amino acid sequence set forth in FIG. 2A (RAF-1). The term “RAF polypeptide” includes a polypeptide that comprises an amino acid sequence having at least about 75%, at least about 80%, at least 85%, at least about 90%, at least about 95%, at least about 98%, at least about 99% or 100% sequence identity to the amino acid sequence set forth in FIG. 2B (A-RAF).

The term “RAF polypeptide” includes a polypeptide that comprises an amino acid sequence having at least about 75%, at least about 80%, at least 85%, at least about 90%, at least about 95%, at least about 98%, at least about 99% or 100% sequence identity to the B-RAF amino acid sequence set forth in FIG. 2C (B-RAF).

Three conserved domains have been identified for the three RAF isoforms: two (CR1 and CR2) in the N terminus and a third (CR3-encoding the serine/threonine kinase domain) in the C terminus. In the regulatory domain, CR1 contains a RAS-binding domain (RBD) and a cysteine-rich domain (CRD), CR2 is a serine/threonine rich domain, and CR3 contains the kinase domain which is involved in RAF activity. See, for example, Srikala et al. (2005) Mol. Cancer Ther. 4(4): 677-685. See also, Bondeva et al. (2002) Molecular Biology of the Cell 13:2323-2333, indicating that the RBD has been narrowed to residues 51-131 within the N-terminal regulatory domain of the RAF-1 molecule. Bondeva et al. also indicate that the CRD domain of RAF-1 (residues 139-184) plays a role in RAS-RAF interaction and creates an additional RAS binding site.

MEK: As used herein, the term “MEK polypeptide” refers to a MAPKK (MAPK kinase) polypeptide having Ser/Thr protein kinase activity and having a molecular weight of about 45 kDa. Two mammalian MEK proteins have been identified, MEK-1 and MEK-2. These proteins are often referred to in the art as MEK-1/2, and this terminology may be used herein when referring to both proteins. MEK polypeptides exhibit kinase activity toward ERK polypeptides, e.g., a MEK polypeptide phosphorylates an ERK polypeptide.

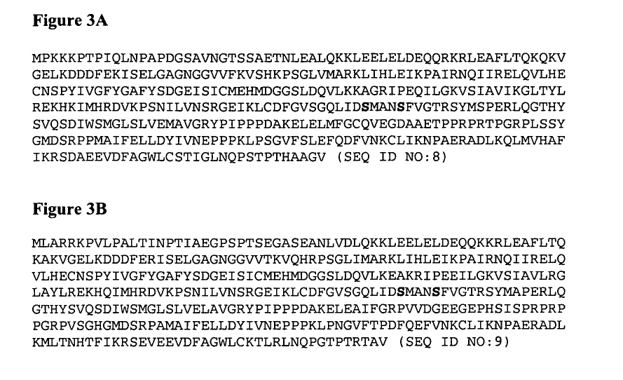

The term “MEK polypeptide” includes a polypeptide that comprises an amino acid sequence having at least about 75%, at least about 80%, at least 85%, at least about 90%, at least about 95%, at least about 98%, at least about 99% or 100% sequence identity to the amino acid sequence set forth in FIG. 3A (MEK-1). The term “MEK polypeptide” includes a polypeptide that comprises an amino acid sequence having at least about 75%, at least about 80%, at least 85%, at least about 90%, at least about 95%, at least about 98%, at least about 99% or 100% sequence identity to the MEK-2 amino acid sequence set forth in FIG. 3B (MEK-2).

ERK: As used herein, the term “ERK polypeptide” refers to a MAPK (MAP kinase) polypeptide having Ser/Thr protein kinase activity, and having a molecular weight of from about 42 kDa to about 44 kDa. Two closely related ERK isoforms of 44 kDa and 42 kDa have been identified. These two isoforms correspond to ERK-1 and ERK-2 respectively. These proteins are often referred to in the art as ERK-1/2, and this terminology may be used herein when referring to both proteins. An ERK polypeptide phosphorylates a serine or a threonine that is next to a proline in a substrate polypeptide. Substrate polypeptides for ERK phosphorylation include, e.g., a transcription factor, e.g., Elk-1; and a cytoplasmic polypeptide such as serine-threonine kinase RSK.

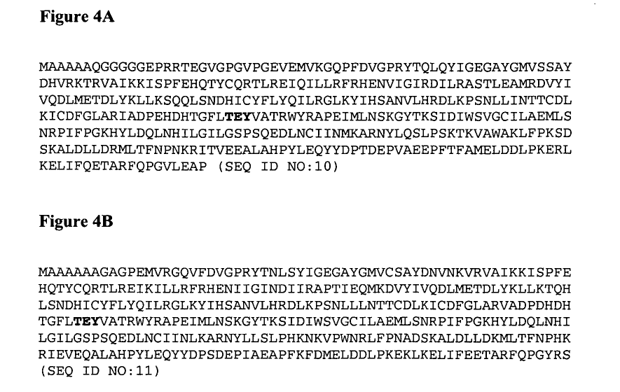

The term “ERK polypeptide” includes a polypeptide that comprises an amino acid sequence having at least about 75%, at least about 80%, at least 85%, at least about 90%, at least about 95%, at least about 98%, at least about 99% or 100% sequence identity to the ERK-1 amino acid sequence set forth in FIG. 4A (ERK-1). The term “ERK polypeptide” includes a polypeptide that comprises an amino acid sequence having at least about 75%, at least about 80%, at least 85%, at least about 90%, at least about 95%, at least about 98%, at least about 99% or 100% sequence identity to the ERK-2 amino acid sequence set forth in FIG. 4B (ERK-2).

The closely related ERK-1 and ERK-2 proteins each comprise a conserved T-E-Y (Thr-Glu-Tyr) activation motif. Full activation of ERK-1 and ERK-2 is achieved via dual phosphorylation at the threonine and tyrosine residues of the motif.

### RAS-RAF-MEK-ERK Pathway Inhibitors

A subject method generally involves administering to an individual in need thereof an effective amount of a RAS-RAF-MEK-ERK pathway inhibitor. As used herein the term “RAS-RAF-MEK-ERK pathway inhibitor” refers to an agent that reduces the activity level of one or more of the following: a RAS polypeptide, a RAF polypeptide, a MEK polypeptide and an ERK polypeptide.

The term “RAS inhibitor” is used herein to refer to an agent that inhibits the expression level and/or activity of a RAS polypeptide. The term “RAS inhibitor” encompasses any agent that prevents proper localization of RAS in the cell membrane, targets the active form of RAS by dislodging it from the cell membrane, or inhibits signaling by RAS to downstream effectors in the RAS-RAF-MEK-ERK signal transduction pathway. In some embodiments, a suitable RAS inhibitor is one that selectively inhibits RAS activity, e.g., the RAS inhibitor selectively inhibits a GTPase activity of RAS and/or selectively inhibits binding of RAS to a RAF polypeptide, where “selective inhibition” means that the inhibitor does not substantially inhibit an activity of a polypeptide other than a RAS polypeptide.

The term “RAF inhibitor” is used herein to refer to an agent that inhibits the expression level and/or activity of a RAF polypeptide. The term “RAF inhibitor” encompasses any agent that inhibits signaling by RAF to downstream effectors in the RAS-RAF-MEK-ERK signal transduction pathway. In some embodiments, a suitable RAF inhibitor is one that selectively inhibits RAF activity, e.g., the RAF inhibitor selectively inhibits serine kinase activity of RAF in phosphorylating a MEK polypeptide (e.g., phosphorylating residues 217 and/or 221 of a MEK polypeptide), where “selective inhibition” means that the inhibitor does not substantially inhibit an activity of a polypeptide other than a RAF polypeptide.

The term “MEK inhibitor” is used herein to refer to an agent that inhibits the expression level and/or activity of a MEK polypeptide. The term “MEK inhibitor” encompasses any agent that inhibits signaling by MEK to downstream effectors in the RAS-RAF-MEK-ERK signal transduction pathway. In some embodiments, a suitable MEK inhibitor is one that selectively inhibits MEK activity, e.g., the MEK inhibitor selectively inhibits phosphorylation of an ERK polypeptide by a MEK polypeptide, where “selective inhibition” means that the inhibitor does not substantially inhibit an activity of a polypeptide other than a MEK polypeptide.

The term “ERK inhibitor” is used herein to refer to an agent that inhibits the expression level, activity and/or downstream signaling of an ERK polypeptide. In some embodiments, a suitable ERK inhibitor is one that selectively inhibits ERK activity, e.g., the ERK inhibitor selectively inhibits proline-directed serine/threonine kinase activity of an ERK polypeptide, where “selective inhibition” means that the inhibitor does not substantially inhibit an activity of a polypeptide other than an ERK polypeptide.

RAS inhibitors: A variety of RAS inhibitors are known in the art which can be used in connection with the methods disclosed herein. Generally, these inhibitors can be divided into four groups based on their mechanism of action: (1) competitive inhibitors of farnesyl PPi, (2) peptidomimetic inhibitors based on the CAAX motif, (3) bisubstrate inhibitors, and (4) inhibitors with unknown mechanisms. CAAX peptidomimetics can either function as alternative substrates in the protein farnesyltransferase (FTase) catalyzed reaction, or they can competitively inhibit FTase without serving as substrates. Exemplary RAS inhibitors are provided below in Table 1. These inhibitors are available from CALBIOCHEM™ a brand of EMD Chemicals, Inc., P.O. Box 12087, La Jolla, Calif. 92039-2087 (USA).

TABLE 1

| Chemical Name/Peptide Seq. | Molecular Formula/Structure |
|---|---|
| 5'-Deoxy-5'-methylthioadenosine | 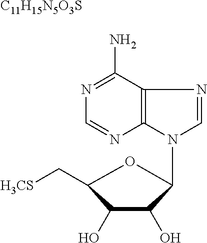 |
| (E,E)-2- [(Dihydroxyphosphinyl)methyl]-3-oxo- 3-[(3,7,11-trimethyl-2,6,10- dodecatrienyl)-amino]propanoic Acid, 3Na | 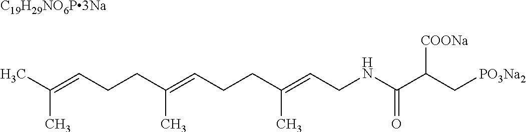 |
| (E,E)-2-[2-Oxo-2-[[(3,7,11-trimethyl- 2,6,10-dodecatrienyl)oxy]amino]ethyl] phosphonic Acid, 2Na | 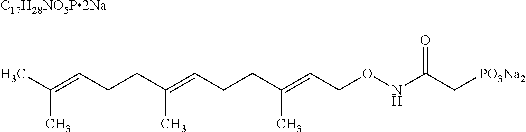 |
| (E,E)-[2-Oxo-2-[[(3,7,11-trimethyl- 2,6,10-dodecatrienyl)oxy]amino]ethyl] phosphonic Acid, (2,2-Dimethyl-1- oxopropoxy)methyl Ester, Na | 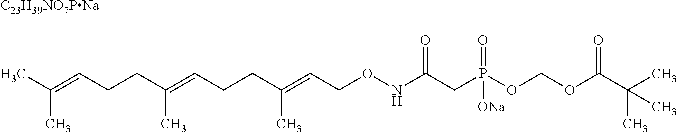 |
| N-[2(S)-[2(R)-Amino-3- mercaptopropylamino]-3-methylbutyl]- Phe-Met-OH B581 | 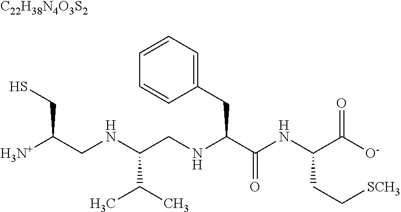 |
| H-Cys-4-Abz-Met-OH | 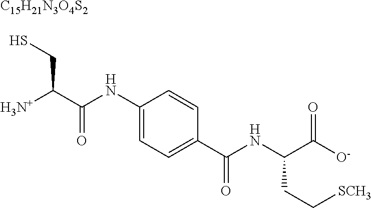 |
| H-Cys-Val-2-Nal-Met-OH(Nal = 2- naphthylalanine) | 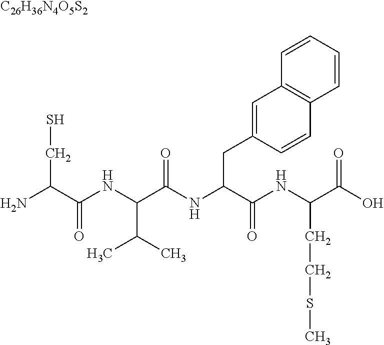 |
| 2-(((5-((1H-Imidazol-4-ylmethyl)- amino)-methyl)-2'-methyl-biphenyl-2- carbonyl)-amino)-4-methylsulfanyl- butyric acid, 2TFA | 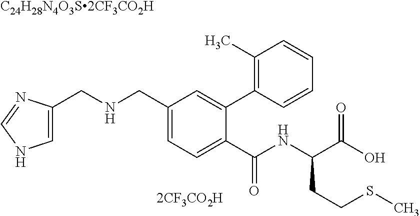 |
| 2-(((5-((1H-Imidazol-4-ylmethyl)- amino)-methyl)-2'-methyl-biphenyl-2- carbonyl)-amino)-4-methylsulfanyl- butyric acid benzyl ester | 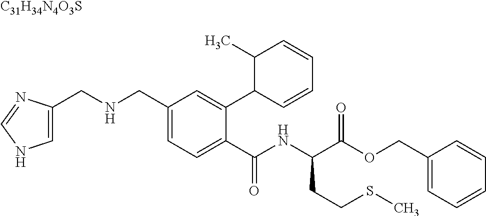 |
| N-[2-pheny]-4-N[2(R)-amino-3- mercaptopropylamino benzoyl]- methionine, TFA | 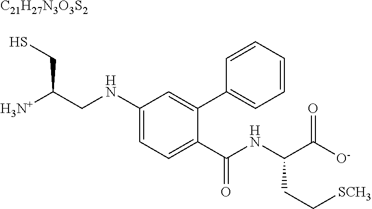 |
| Methyl {N-[2-phenyl-4-N[2(R)-amino- 3-mecaptopropylamino]benzoyl]}- methionate, TFA | 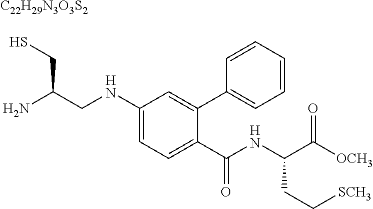 |
| N-4-[2(R)-Amino-3- mercaptopropyl]amino-2- phenylbenzoyl-(L)-leucine methyl ester, TFA | 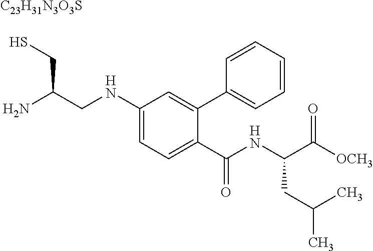 |
| N-4-[2(R)-Amino-3- mercaptopropyl]amino-2- phenylbenzoyl-(L)-leucine,TFA | 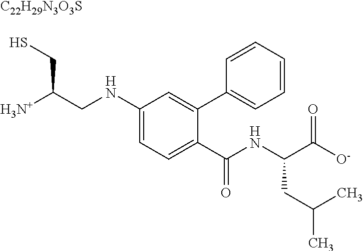 |
| N-4-[2(R)-Amino-3- mercaptopropyl]amino-2- naphthylbenzoyl-(L)-leucine, TFA | 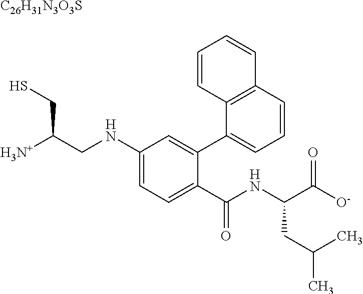 |
| 2,3,5a,6-Tetrahydro-6-hydroxy- 3(hyroxymethyl)-2-methyl-10H-3a,10a- epidithio-pyrazinol[1,2a]indole-1,4- dione | 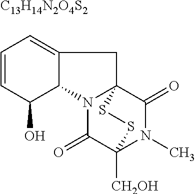 |
| | 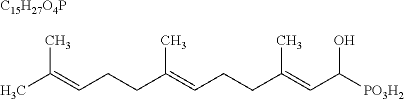 |

An additional RAS inhibitor, Farnesylthiosalicylic acid (C₂₂H₃₀O₂S), has the molecular structure provided below.

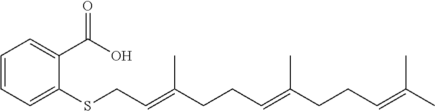

Zarnestra (also known as R115777; (+)-6-[amino(4-chlorophenyl)(1-methyl-1H-imidazol-5-yl)methyl]-4-(3-chlorophenyl)-1-methyl-2(1H)-quinolinone), is suitable for use in a subject method. Suitable dosages of R115777 include 300 mg twice daily, administered orally, and 600 mg twice daily, administered orally.

RAS inhibitors are also described in U.S. Patent Application Publication 20070054886, published Mar. 8, 2007, which publication is incorporated by reference herein for its disclosure of various RAS inhibitors. RAS inhibitors include farnesyl protein transferase (FPT) inhibitors, e.g., FPT III ((E,E)-[2-oxo-2-[[(3,7,11-trimethyl-2,6,10-dodecatrienyl)oxy]amino]ethyl]phosphonic Acid, (2,2-Dimethyl-1-oxopropoxy)methyl Ester, Na).

RAF inhibitors: A variety of RAF inhibitors are known in the art which can be used in connection with the methods disclosed herein. These include, for example, GW 5074, BAY 43-9006, ISIS 5132 and ZM 336372. GW 5074 (available from Tocris Bioscience, 16144 Westwoods Business Park, Ellisville, Mo., 63021, USA) has been shown to inhibit RAF-1 kinase activity in vitro with an IC₅₀ of 9 nM. BAY 43-9006 (also known as Sorafenib™), has been shown to inhibit RAF-1 kinase activity in vitro with an IC₅₀ of 12 nM. ISIS 5132, a 20-base phosphorothioate antisense oligodeoxynucleotide designed to hybridize to the 3′ untranslated region of the c-raf-1 mRNA, has been shown to inhibit c-raf-1 expression in culture with an IC₅₀ between 50 and 100 nM. See, e.g., Kohno and Pouyssegur (2003) Progress in Cell Cycle Research 5: 219-224. ZM 336372, a potent ATP-competitive inhibitor of RAF-1 in vitro (IC₅₀=70 nM), is available from Cayman Chemical Company, 1180 East Ellsworth Road, Ann Arbor, Mich. 48108.

A variety of RAF-1 inhibitors, including BAY 43-9006, are described in U.S. Patent Application Publication No. 2008/0032979, published Feb. 7, 2008, and International Patent Application Publication No. WO/2000/042012, published Jul. 20, 2000, which applications are incorporated by reference herein for their disclosure of RAF-1 inhibitors. The molecular structures for GW 5074, BAY 43-9006, and ZM 336372 are provided below in Table 2.

TABLE 2

| | |
|---|---|
| GW 5074 | 3-(3,5-Dibromo-4-hydroxy-benzylidene)-5-iodo-1,3-dihydro-indol-2-one |
| | 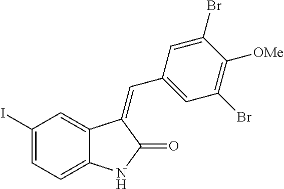 |
| BAY 43-9006 | 4-[4-[[4-chloro-3-(trifluoromethyl)phenyl]carbamoylamino]phenoxy]- N-methyl-pyridine-2-carboxamide |
| | 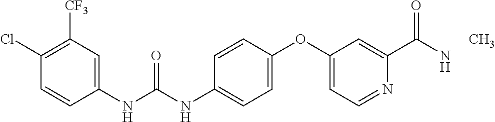 |
| ZM 336372 | N-[5-(3-Dimethylaminobenzamido)-2-methylphenyl]-4-hydroxybenzamide |
| | 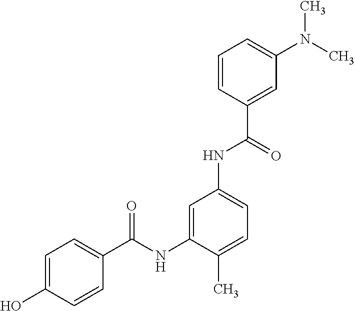 |

Suitable dosages are known in the art or can be readily determined by those of ordinary skill in the art. For example, BAY 43-9006 can be administered at a dosage of from 200 mg twice daily to 400 mg twice daily, where administration is oral.

MEK 1/2 inhibitors: Specific and potent MEK 1/2 inhibitors include PD98059 (2′-amino-3′-methoxyflavone), U0126 and PD184352. See, e.g., Kohno and Pouyssegur (2003) Progress in Cell Cycle Research 5: 219-224; See also, Ahn et al. (1999) Promega Notes, Number 71, page 4 (publication available from Promega Corp., 2800 Woods Hollow Road, Madison Wis., 53711, USA). PD184352 (also known as CI-1040) is described in Sebolt-Leopold (2000) Oncogene 19: 6594-6599.

An additional potent MEK 1/2 inhibitor, PD198306 (available from Tocris Bioscience, 16144 Westwoods Business Park, Ellisville, Mo. 63021, USA) inhibits isolated enzyme at a concentration of 8 nM and inhibits Mek activity in synovial fibroblasts at concentrations of 30-100 nM. This compound is highly selective for MEK, with IC₅₀ values >1, >4, >4 and >10 μM for ERK, c-Src, cdks and PI 3-kinase γ respectively.

Wyeth-Ayerst has developed a series of 3-cyano-4-(phenoxyanilino) quinolines as MEK inhibitors. Of these, Compound 14 is the most potent, inhibiting MEK-1 activity in vitro with an IC₅₀ of 2.4 nM. See, e.g., Zhang et al. (2000) Bioorg. Med. Chem. Lett. 10: 2825-2828. Potent inhibitory activity towards Mek has also been demonstrated for resorcylic acid lactones isolated from microbial extracts. For example, Ro 09-2210, isolated from a fungal broth FC2506 inhibits MEK-1 activity in vitro with an IC₅₀ of 60 nM. Williams et al. (1998) Biochemistry 37: 9579-9585. Another compound, L-783,277, purified from organic extracts of Phoma sp. (ATCC 74403) inhibited MEK-1 activity in vitro with an IC₅₀ of 4 nM. Zhao et al. (1999) J. Antibiot. 52: 1086-1094. The molecular structures for PD98059, U0126, PD184352, PD198306, Compound 14, Ro 09-2210 and L-783,277 are provided below in Table 3.

TABLE 3

| | |
|---|---|
| PD98059 | 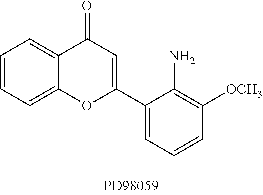 |
| U0126 | 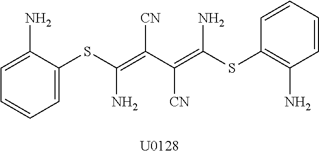 |
| PD184352 | 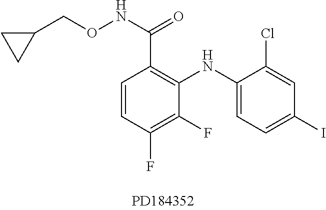 |
| PD198306 | N-(Cyclopropylmethoxy)-3,4,5-trifluoro-2-[(4-iodo-2- methylphenyl)amino]-benzamide |
| | 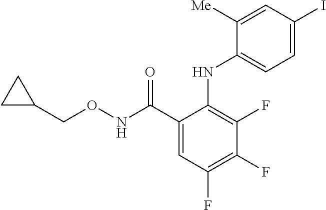 |
| Compound 14 | 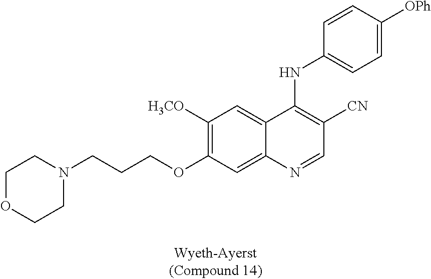 |
| Ro 09-2210 | 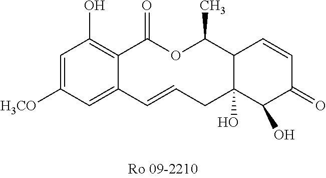 |
| L-783,277 | 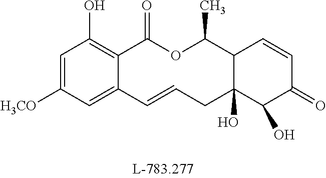 |

Peptide drugs have also been identified as MEK-1 inhibitors. For example, a peptide corresponding to the amino-terminal amino acid sequence MPKKKPTPIQLNP (SEQ ID NO:12) of MEK-1, a region involved in the association of ERK 1/2 with MEK-1, has been shown to specifically inhibit the activation of ERK 1/2. See, e.g., Kelemen et al. (2002) J. Biol. Chem. 277: 8741-8748. To allow for efficient entry of peptide into cells, the peptide can be modified with membrane-translocating moieties. For example, the inhibitor peptide can be modified via alkylation (myristoylation or stearation) in order to increase its hydrophobicity and hence its cellular uptake. The inhibitor peptide can also be modified by linking to a membrane-translocating peptide (MTP) to facilitate the cellular delivery of the peptide. Several MTPs, capable of transporting peptides or even large proteins, have been described recently (Schwarze et al. (2000) Trends Cell Biol. 10: 290-295). These include peptides derived from the Drosophila melanogaster antennapedia (Antp) homeotic transcription factor (Derossi et al. (1994) J. Biol. Chem. 269: 10444-10450), the human immunodeficiency virus-TAT (TAT) protein (Fawell et al. (1994) Proc. Natl. Acad. Sci. U.S.A. 91: 664-668), the h region of the signal sequence of Kaposi fibroblast growth factor (MTS) (Lin et al. (1995) J. Biol. Chem. 270: 14255-14258), and the protein PreS2 of hepatitis B virus (HBV) (Oess, S., and Hildt, E. (2000) Gene Ther. 7: 750-758). Inclusion of either an alkyl moiety or a membrane-translocating peptide sequence was shown to facilitate the cellular uptake of the peptide inhibitor and prevent ERK activation in phorbol ester-stimulated NIH3T3 cells and NGF-treated PC12 cells with an IC₅₀ of 13˜45 μM.

An additional peptide capable of inhibiting the activation of ERK by MEK is available from Imgenex Corp., 11175 Flintkote Avenue, Suite E, San diego, CA, 92121 (USA). This peptide has the amino acid sequence DRQIKIWFQNRRMKWKKGMPKKKPTPIQLN (SEQ ID NO:13), wherein the underlined portion is the inhibitor sequence which blocks the activation of ERK by Mek and the DRQIKIWFQNRRMKWKK (SEQ ID NO:14) sequence is a protein transduction (PTD) sequence derived from antennapedia which renders the peptide cell permeable.

ERK 1/2 inhibitors: Specific inhibitors of ERK 1/2 have also been described. For example, FR180204 (5-(2-phenylpyrazolo[1,5-a]pyridin-3-yl)-1H-pyrazolo[3,4-c]pyridazin-3-amine), has been shown to inhibit the kinase activity of ERK1 and ERK2, with K(i) values 0.31 and 0.14 μM, respectively. See, e.g., Ohori et al. (2005) Biochem Biophys Res Commun. 336(1):357-63. Hypericin, an aromatic polycyclic dione isolated from plants of the Hypericum family exhibits an IC₅₀ of 4 nM for ERK-2. See, e.g., Jacque et al. (1998) EMBO J. 17: 2607-2618. The molecular structures for FR180204 and hypericin are provided below in Table 4.

TABLE 4

| | |
|---|---|
| FR180204 | 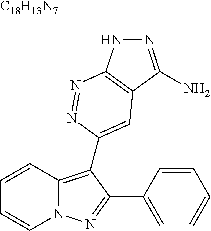 |
| hypericin | 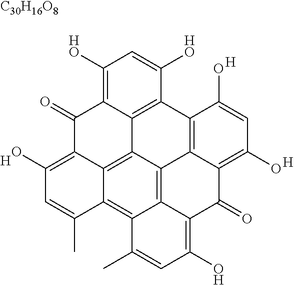 |

Additional inhibitors of MEK 1/2 and/or ERK 1/2 are available from CALBIOCHEM™ a brand of EMD Chemicals, Inc., P.O. Box 12087, La Jolla, Calif. 92039-2087 (USA). The catalog numbers and identifying information for these compounds are indicated in Table 5 below.

TABLE 5

| Chemical Name/Peptide Seq. | Molecular Formula/Structure |
|---|---|
| 4',5,7-Trihydroxyflavone | 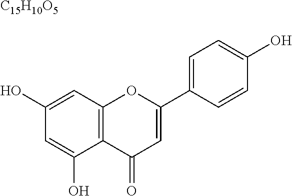 |
| Ste-MPKKKPTPIQLNP-NH₂ (SEQ ID NO: 12) (Ste = stearated) | |
| H-GYGRKKRRQRRR-G- MPKKKPTPIQLNP-NH₂ (SEQ ID NO: 15) | |
| 3-(2-Aminoethyl)-5-((4- ethoxyphenyl)methylene)-2,4- thiazolidinedione, HCl | 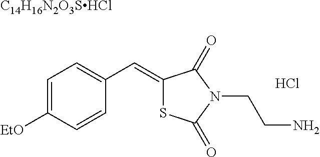 |
| 5-(2-Phenyl-pyrazolo[1,5-a]pyridin-3-yl)- 1H-pyrazolo[3,4-c]pyridazin-3-ylamine (a.k.a. FR180204) | 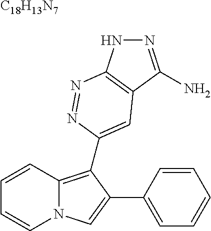 |
| 4-Amino-5-iodo-7-(β-D- ribofuranosyl)pyrrolo[2,3-d]pyrimidine | 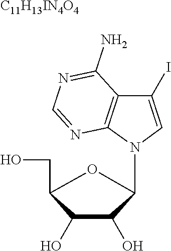 |
| | 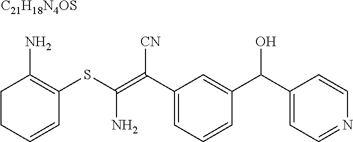 |
| | 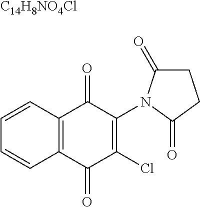 |
| Z-& E-α-(Amino-((4- aminophenyl)thio)methylene)-2- (trifluoromethyl)benzeneacetonitrile | 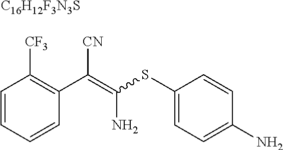 |
| 2'-Amino-3'-methoxyflavone (a.k.a PD 98059) | 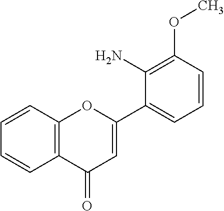 |
| 1,4-Diamino-2,3-dicyano-1,4 bis(phenylthio)butadiene (a.k.a. U0125) | 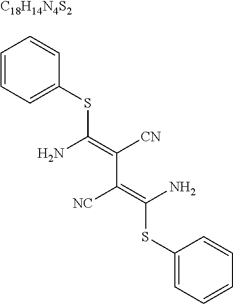 |
| 1,4-Diamino-2,3-dicyano-1,4-bis(2- aminophenylthio)butadiene (a.k.a U0126) | 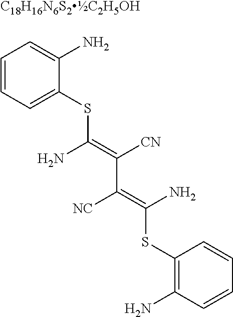 |
| α-Cyano-(3-hydroxy-4- nitro)cinnamonitrile | 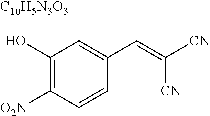 |

### Combination Therapies

In some embodiments, a subject method involves administration of two or more RAS-RAF-MEK-ERK pathway inhibitors. In some embodiments, an inhibitor of a RAS-RAF-MEK-ERK pathway is administered in combination therapy with at least one additional suitable therapeutic agent. Thus, in some embodiments, a subject method involves administering: a) an agent that inhibits an activity of a RAS-RAF-MEK-ERK pathway member; and b) at least one additional therapeutic agent. In some embodiments, the at least one additional therapeutic agent is an anti-viral agent, e.g., an agent that has activity in inhibiting HCV.

In some embodiments, the at least one additional therapeutic agent is a p38 MAPK inhibitor. Suitable p38 MAPK inhibitors include, e.g., SB203580 (4-(4-Fluorophenyl)-2-(4-methylsulfinylphenyl)-5-(4-pyridyl)-1H-imidazole).

In some embodiments, the at least one additional therapeutic agent includes interferon-alpha (IFN-α). Any known IFN-α can be used in a combination therapy. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con₁ is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods.

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

Effective dosages of Infergen™ consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b range from 3 million Units (MU) to 10 MU per dose. Effective dosages of PEGASYS™PEGylated IFN-α2a contain an amount of about 90 μg to 270 μg, or about 180 μg, of drug per dose. Effective dosages of PEG-INTRON™ PEGylated IFN-α2b contain an amount of about 0.5 μg to 3.0 μg of drug per kg of body weight per dose. Effective dosages of PEGylated consensus interferon (PEG-CIFN) contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN contain an amount of about 45 μg to about 270 or about 60 μg to about 180 μg, or about 90 μg to about 120 μg, of drug per dose. IFN-α can be administered daily, every other day, once a week, three times a week, every other week, three times per month, once monthly, substantially continuously or continuously.

In some embodiments, the at least one additional suitable therapeutic agent includes ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin may be administered orally in capsule or tablet form, or in the same or different administration form and in the same or different route as the RAS-RAF-MEK-ERK pathway inhibitor. Of course, other types of administration of both medicaments, as they become available are contemplated, such as by nasal spray, transdermally, by suppository, by sustained release dosage form, etc. Any suitable form of administration can be utilized so long as the proper dosages are delivered without destroying the active ingredient.

Ribavirin can be administered in an amount ranging from about 400 mg to about 1200 mg, from about 600 mg to about 1000 mg, or from about 700 to about 900 mg per day. In some embodiments, ribavirin is administered throughout the entire course of therapy with a RAS-RAF-MEK-ERK pathway inhibitor. In other embodiments, ribavirin is administered only during a first period of time. In still other embodiments, ribavirin is administered only during a second period of time.

### Levovirin

In some embodiments, the at least one additional suitable therapeutic agent includes levovirin. Levovirin is the L-enantiomer of ribavirin. Levovirin is manufactured by ICN Pharmaceuticals.

Levovirin has the following structure:

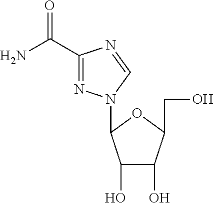

### Viramidine

In some embodiments, the at least one additional suitable therapeutic agent includes viramidine. Viramidine is a 3-carboxamidine derivative of ribavirin, and acts as a prodrug of ribavirin. It is efficiently converted to ribavirin by adenosine deaminases.

Viramidine has the following structure:

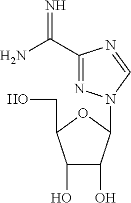

Nucleoside analogs that are suitable for use in a subject combination therapy include, but are not limited to, ribavirin, levovirin, viramidine, isatoribine, an L-ribofuranosyl nucleoside as disclosed in U.S. Pat. No. 5,559,101 and encompassed by Formula I of U.S. Pat. No. 5,559,101 (e.g., 1-β-L-ribofuranosyluracil, 1-β-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuranosylguanine, 9-β-L-ribofuranosyl-6-thioguanine, 2-amino-α-L-ribofuran[1′,2′:4,5]oxazoline, O²,O²-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofuranosyl-5-fluorouracil, 2-amino-β-L-arabinofurano[1′,′:4,5]oxazoline, O²,O²-anhydro-β-L-arabinofuranosyluracil, 2′-deoxy-β-L-uridine, 3′5′-Di-O-benzoyl-2′deoxy-4-thio β-L-uridine, 2′-deoxy-β-L-cytidine, 2′-deoxy-β-L-4-thiouridine, 2′-deoxy-O-L-thymidine, 2′-deoxy-β-L-5-fluorouridine, 2′,3′-dideoxy-β-L-uridine, 2% deoxy-β-L-5-fluorouridine, and 2′-deoxy-β-L-inosine); a compound as disclosed in U.S. Pat. No. 6,423,695 and encompassed by Formula I of U.S. Pat. No. 6,423,695; a compound as disclosed in U.S. Patent Publication No. 2002/0058635, and encompassed by Formula 1 of U.S. Patent Publication No. 2002/0058635; a nucleoside analog as disclosed in WO 01/90121 A2 (Idenix); a nucleoside analog as disclosed in WO 02/069903 A2 (Biocryst Pharmaceuticals Inc.); a nucleoside analog as disclosed in WO 02/057287 A2 or WO 02/057425 A2 (both Merck/Isis); and the like.

HCV NS3 inhibitors

In some embodiments, the at least one additional suitable therapeutic agent includes an HCV NS3 inhibitor. Suitable HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301 A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54^{th} Ann. Meeting AASLD); NS3 inhibitor SCH6 (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); an NS3 protease inhibitor as disclosed in any one of U.S. Patent Publication Nos. 2003019067, 20030187018, and 20030186895; and the like.

Of particular interest in many embodiments are NS3 inhibitors that are specific NS3 inhibitors, e.g., NS3 inhibitors that inhibit NS3 serine protease activity and that do not show significant inhibitory activity against other serine proteases such as human leukocyte elastase, porcine pancreatic elastase, or bovine pancreatic chymotrypsin, or cysteine proteases such as human liver cathepsin B.

### NS5B Inhibitors

In some embodiments, the at least one additional suitable therapeutic agent includes an NS5B inhibitor. Suitable HCV non-structural protein-5 (NS5; RNA-dependent RNA polymerase) inhibitors include, but are not limited to, a compound as disclosed in U.S. Pat. No. 6,479,508 (Boehringer-Ingelheim); a compound as disclosed in any of International Patent Application Nos. PCT/CA02/01127, PCT/CA02/01128, and PCT/CA02/01129, all filed on Jul. 18, 2002 by Boehringer Ingelheim; a compound as disclosed in U.S. Pat. No. 6,440,985 (ViroPharma); a compound as disclosed in WO 01/47883, e.g., JTK-003 (Japan Tobacco); a dinucleotide analog as disclosed in Zhong et al. (2003) Antimicrob. Agents Chemother. 47:2674-2681; a benzothiadiazine compound as disclosed in Dhanak et al. (2002) J. Biol Chem. 277(41):38322-7; an NS5B inhibitor as disclosed in WO 02/100846 A1 or WO 02/100851 A2 (both Shire); an NS5B inhibitor as disclosed in WO 01/85172 A1 or WO 02/098424 A1 (both Glaxo SmithKline); an NS5B inhibitor as disclosed in WO 00/06529 or WO 02/06246 A1 (both Merck); an NS5B inhibitor as disclosed in WO 03/000254 (Japan Tobacco); an NS5B inhibitor as disclosed in EP 1 256,628 A2 (Agouron); JTK-002 (Japan Tobacco); JTK-109 (Japan Tobacco); and the like.

Of particular interest in many embodiments are NS5 inhibitors that are specific NS5 inhibitors, e.g., NS5 inhibitors that inhibit NS5 RNA-dependent RNA polymerase and that lack significant inhibitory effects toward other RNA dependent RNA polymerases and toward DNA dependent RNA polymerases.

### Exemplary Combination Therapy

In one non-limiting embodiment, BAY 43-9006 is administered orally at a dose of 200 mg twice daily; and PEGASYS™ PEGylated IFN-α2b is administered at 180 μg per dose. In one non-limiting embodiment, BAY 43-9006 is administered orally at a dose of 200 mg twice daily; and Infergen™ consensus IFN-α is administered at about 18 μg per dose. In one non-limiting embodiment, BAY 43-9006 is administered orally at a dose of 200 mg twice daily; and PEG-INTRON™ PEGylated IFN-α2b is administered at about 1.0 μg per kg body weight per dose. In any of these exemplary embodiments, ribavirin can be administered in an amount of from about 400 mg to about 1200 mg per day.

In another non-limiting embodiment, R115777 is administered orally in an amount of 300 mg twice daily; and PEGASYS™ PEGylated IFN-α2b is administered at 180 μg per dose. In one non-limiting embodiment, R115777 is administered orally in an amount of 300 mg twice daily; and Infergen™ consensus IFN-α is administered at about 18 μg per dose. In one non-limiting embodiment, R115777 is administered orally in an amount of 300 mg twice daily; and PEG-INTRON™ PEGylated IFN-α2b is administered at about 1.0 μg per kg body weight per dose. In any of these exemplary embodiments, ribavirin can be administered in an amount of from about 400 mg to about 1200 mg per day.

In another non-limiting embodiment, PD184352 is administered orally in an amount of 800 mg bid; and PEGASYS™ PEGylated IFN-α2b is administered at 180 μg per dose. In one non-limiting embodiment, PD184352 is administered orally in an amount of 800 mg bid; and Infergen™ consensus IFN-α is administered at about 18 μg per dose. In one non-limiting embodiment, PD184352 is administered orally in an amount of 800 mg bid; and PEG-INTRON™ PEGylated IFN-α2b is administered at about 1.0 μg per kg body weight per dose. In any of these exemplary embodiments, ribavirin can be administered in an amount of from about 400 mg to about 1200 mg per day.

### Formulations, Dosages, and Routes of Administration

An agent that inhibits a RAS-RAF-MEK-ERK pathway (referred to herein as an “active agent”) can be formulated in a variety of ways suitable for administration. An active agent can be provided in combination with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an active agent is formulated in an aqueous or non-aqueous formulation, which may further include a buffer. Suitable aqueous buffers include, but are not limited to, acetate, succinate, citrate, and phosphate buffers varying in strength from 5 mM to 100 mM. In some embodiments, the aqueous buffer includes reagents that provide for an isotonic solution. Such reagents include, but are not limited to, sodium chloride, and sugars e.g., mannitol, dextrose, sucrose, and the like. In some embodiments, the aqueous buffer further includes a non-ionic surfactant such as polysorbate 20 or 80.

Optionally the formulations may further include a preservative. Suitable preservatives include, but are not limited to, a benzyl alcohol, phenol, chlorobutanol, benzalkonium chloride, and the like. In some cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In the subject methods, the active agents may be administered to the host using any convenient means capable of resulting in the desired therapeutic effect. Thus, the agents can be incorporated into a variety of formulations for therapeutic administration. For example, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an active agent may be administered in the form of its pharmaceutically acceptable salts, or an active agent may also be used alone or in appropriate association, as well as in combination, with other pharmaceutically active compounds. The following methods and excipients are merely exemplary and are in no way limiting.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature. An active agent can also be provided in sustained release or controlled release formulations, e.g., to provide for release of agent over time and in a desired amount (e.g., in an amount effective to provide for a desired therapeutic or otherwise beneficial effect).

Unit dosage forms for oral or rectal administration such as syrups, elixirs, and suspensions may be provided wherein each dosage unit, for example, teaspoonful, tablespoonful, tablet or suppository, contains a predetermined amount of a composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of the agents calculated in an amount sufficient to produce the desired effect in association with a pharmaceutically acceptable diluent, carrier or vehicle. The specifications for the unit dosage forms for use in connection with a subject method depend on the particular compound employed and the effect to be achieved, the pharmacodynamics associated with each compound in the host, and the like.

Dosage forms of particular interest include those suitable to accomplish intravenous or oral administration, as well as dosage forms to provide for delivery by a nasal or pulmonary route (e.g., inhalation), e.g., through use of a metered dose inhaler and the like.

An active agent can be formulated in either parenteral or enteral forms, in some embodiments enteral formulations, e.g., oral formulations. An active agent can be formulated for parenteral administration, e.g., by subcutaneous, intradermal, intraperitoneal, intravenous, or intramuscular injection. Administration may also be accomplished by, for example, enteral, oral, buccal, rectal, transdermal, intratracheal, inhalation (see, e.g., U.S. Pat. No. 5,354,934), etc.

An active agent can be administered in an amount of from about 10 mg to about 1000 mg per dose, e.g., from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

In some embodiments, the amount of an active agent per dose is determined on a per body weight basis. For example, in some embodiments, an active agent is administered in an amount of from about 0.5 mg/kg to about 50 mg/kg, e.g., from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, or from about 40 mg/kg to about 50 mg/kg per dose. In other embodiments, an active agent is administered in an amount of from about 5 mg/kg to about 100 mg/kg, e.g., from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg per dose.

Those of skill will readily appreciate that dose levels can vary as a function of the specific compound, the severity of the symptoms and the susceptibility of the subject to side effects. Preferred dosages for a given active agent are readily determinable by those of skill in the art by a variety of means.

In some embodiments, multiple doses of an active agent are administered. The frequency of administration of an active agent can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, an active agent is administered continuously.

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, an active agent is administered for the lifetime of the individual.

In some embodiments, administration of an active agent is discontinuous, e.g., an active agent is administered for a first period of time and at a first dosing frequency; administration of the active agent is suspended for a period of time; then the active agent is administered for a second period of time for a second dosing frequency. The period of time during which administration of the active agent is suspended can vary depending on various factors, e.g., blood glucose levels; and will generally range from about 1 week to about 6 months, e.g., from about 1 week to about 2 weeks, from about 2 weeks to about 4 weeks, from about one month to about 2 months, from about 2 months to about 4 months, or from about 4 months to about 6 months, or longer. The first period of time may be the same or different than the second period of time; and the first dosing frequency may be the same or different than the second dosing frequency.

### Subjects Suitable for Treatment

Individuals that are suitable for treatment with a subject method for treating an HCV infection include individuals who have been diagnosed with an HCV infection. Any of the above treatment regimens can be administered to individuals who have been diagnosed with an HCV infection. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naive individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased.

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Individuals that are suitable for treatment with a subject method for treating an HCV infection include individuals who have an HCV infection and, as a result of the HCV infection, suffer from liver fibrosis. Such individuals include HCV-infected individuals as described above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### RAS-RAF-MEK-ERK Pathway Inhibitors Decrease Levels of HCV RNA in Supernatant

### Materials and Methods

Day 0: Huh7.5 cells were transfected with an eGFP-Jc1 reporter construct RNA via electroporation (10 μg RNA per 4×10⁶ cells). The eGFP-Jc1 reporter construct is a modified version of the Jc1 (J6/C3 chimera) described by Pietschmann et al. (2006) PNAS 103(19): 7408-7413, in which an eGFP (enhanced green fluorescent protein)-IRES (internal ribosomal entry site) cassette has been inserted between the 5′UTR and the open reading frame of the Jc1 construct.

Transfected cells were then plated at 2×10⁶ cells per T75 flask.

Day 1: The cells were washed 3 times and treated with 10 μl dimethylsulfoxide (DMSO) (control), 10 μM ERK Inhibitor II (FR180204Calbiochem) or 10 μM U0126 (Promega).

Day 3: The transfected Huh7.5 cells and the supernatant were harvested. The harvested cells and the supernatant were analyzed by quantitative reverse transcription-polymerase chain reaction (qRT-PCR) for levels of HCV RNA

Total RNA was extracted from transfected cells using RNA STAT-60 REAGENT (Tel Test).

Viral RNA in culture supernatants was isolated using RNA STAT-50 LS REAGENT (Tel Test). total cellular RNA or 10 μl of RNA of culture supernatant was used for cDNA synthesis using SuperScript III reverse transcriptase (Invitrogen) and random hexamer primers. Viral RNA levels were measured by quantitative real-time PCR using Taqman probes and corresponding primers.

### Results

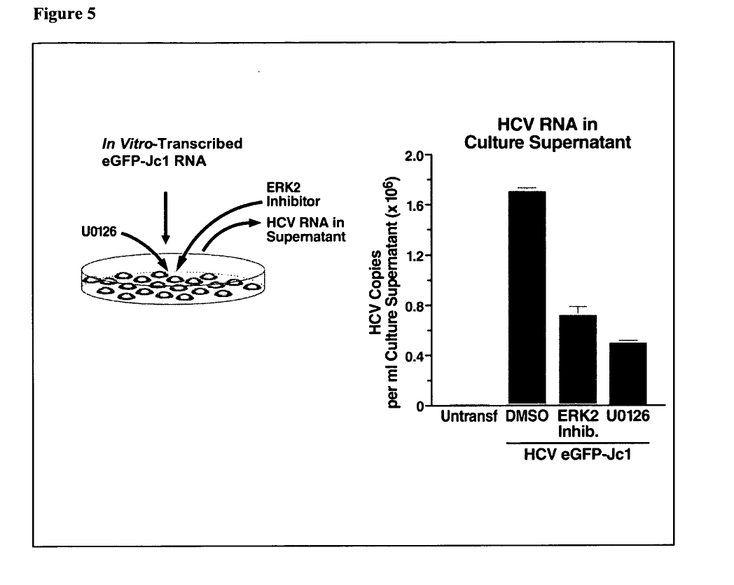

Treatment with ERK Inhibitor II (FR180204) decreased the number of HCV copies per ml supernatant by ˜50% compared with control treated cells. The reduction in infectious particle production was even more pronounced with U0126, an inhibitor of the upstream kinase MEK. The supernatant of U0126 treated cells contained only one third as many HCV copies per ml as the supernatant of control treated cells. A graphical representation of the above results is set forth in FIG. 5.

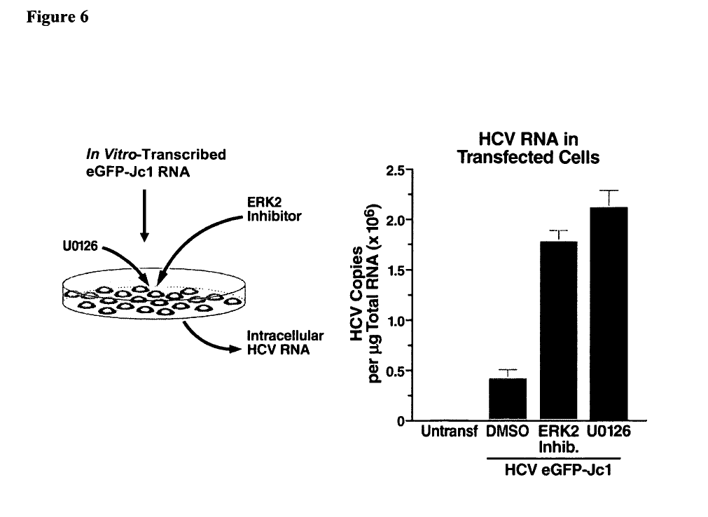

Analysis of the harvested cells by qRT-PCR indicated that the level of intracellular HCV RNA per μg total RNA increased when cells were treated with ERK Inhibitor II and U0126 respectively. This suggests that the inhibition of infectious particle production is not due to decreased RNA replication within the cells. Results of the analysis of intracellular HCV RNA levels are set forth graphically in FIG. 6.

### Example 2

### RAS-RAF-MEK-ERK Pathway Inhibitors Impair Production of Infectious Virus

Materials and Methods

Huh7.5 cells were transfected on Day 0 with eGFP-Jc1 reporter construct RNA via electroporation (10 μg RNA per 4×10⁶ cells). Cells were then plated at 2×10⁶ cells per T75 flask.

On Day 1 the cells were washed 3 times and treated with 10 μl DMSO (control), 10 μM ERK Inhibitor II (FR180204 Calbiochem), 10 μM U0126 (Promega), 10 μM FPT Inhibitor III (RAS inhibitor, see Table 1 above), 50 μM PD98059 (MEK 1/2 inhibitor, see Table 3 above), or 10 μM ZM 336372 (RAF inhibitor, See Table 2 above).

On Day 3 virus particles in the supernatant were filtered & concentrated using an Amicon Ultra MWCO 100 filter (10 ml of supernatant was concentrated to a volume of 200 μl). Huh7.5 cells plated at 5×10⁴ cells per well on Day 2 (24 well plate) were then infected with 50 μl of the concentrated virus per well for 3 h at 37° C. The cells were subsequently washed 3 times.

On Day 5 the infected cells were harvested and subsequently analyzed by FACS.

Results

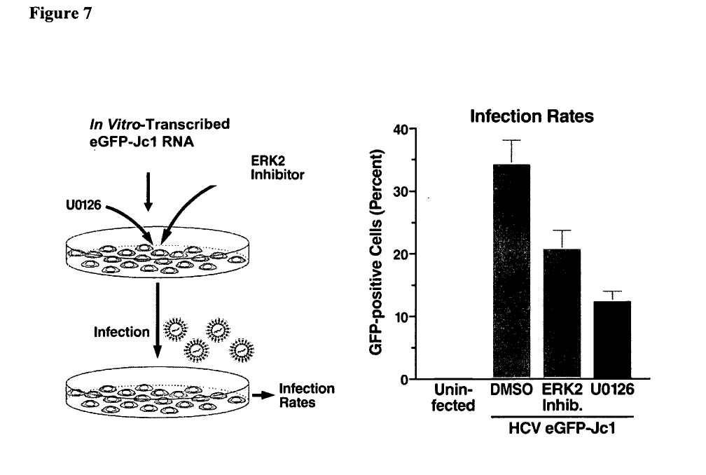

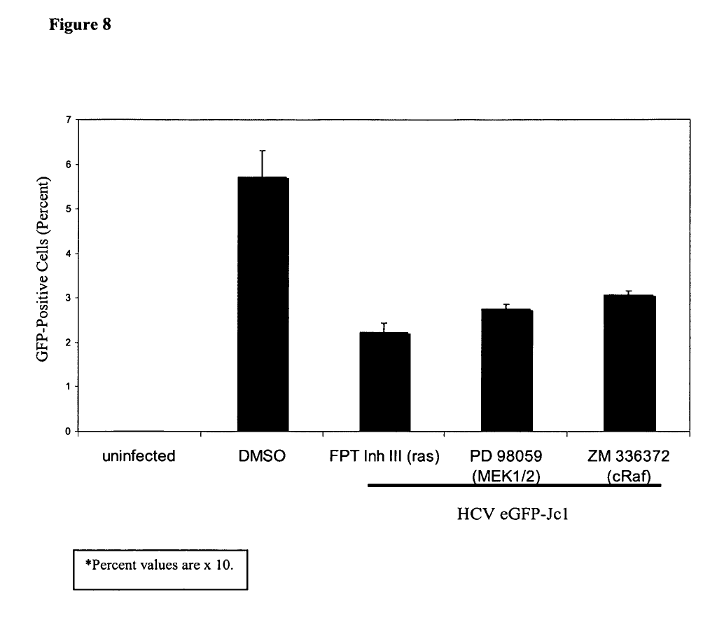

Infection rates, as determined by the percentage of GFP positive cells, were significantly reduced for each of the above inhibitors relative to DMSO control. Graphical representations of these results are shown in FIGS. 7 and 8.

### Example 3

### RAS-RAF-MEK-ERK Pathway Inhibitors Impair Spreading Infection

Materials and Methods

Virus was prepared as follows: Huh7.5 cells were transfected with eGFP-Jc1 reporter construct RNA via electroporation (10 μg RNA per 4×10⁶ cells). After 3-5 days of incubation, virus containing supernatant was harvested. Virus particles in the supernatant were filtered & concentrated using an Amicon Ultra MWCO 100 filter (10 ml of supernatant was concentrated to a volume of 200 μl).

Huh7.5 cells were then infected according to the following protocol.

Day 0: Cells were plated at 1×10⁴ cells per well in 24 well plates.

Day 1: Pretreat cells with 10 μl DMSO, 10 μM ERK Inhibitor II or 10 μM U0126 for 1 hour.

Infection with 50 μl conc. virus per well for 3 h at 37° C.

Addition of 10 μg/ml-CD81 antibody for single-round infection

Day 3/4: Harvest infected cells for FACS analysis.

### Example 4

### HCV Core Expression Induces Hyperphosphorylation of ERK2

HCV core transgenic mice were generated, where the mice were transgenic for an HCV core coding sequence under control of a tetracycline response element (TRE), and transgenic for a tetracycline-regulatable transcriptional activator protein (tTA) coding sequence under control of the liver-specific liver-activator protein (LAP) promoter. Thus, expression of HCV core is repressed in the transgenic mice by addition of tetracycline or doxycycline to the diet of the mice; induction of HCV core expression is achieved by removal of the tetracycline or doxycycline from the diet. Gossen et al. (1995) Science 268:1766; Kistner et al. (1996) Proc. Natl. Acad. Sci. USA 93:10933.

HCV core transgenic mice were kept on a Dox-containing diet. 11 weeks prior to sample preparation, mice were placed on Dox-free diet to induce the expression of HCV Core. Samples from single transgenic mice (only harboring tetracycline-controlled transactivator tTA under the control of the liver specific LAP promoter) and double transgenic mice (tTA and TRE-HCV Core) on and off Dox diet were analyzed by western blot for the phosphorylation state of ERK2 using anti-phospho-ERK1/2 antibodies (α-Phospho-ERK1/2).

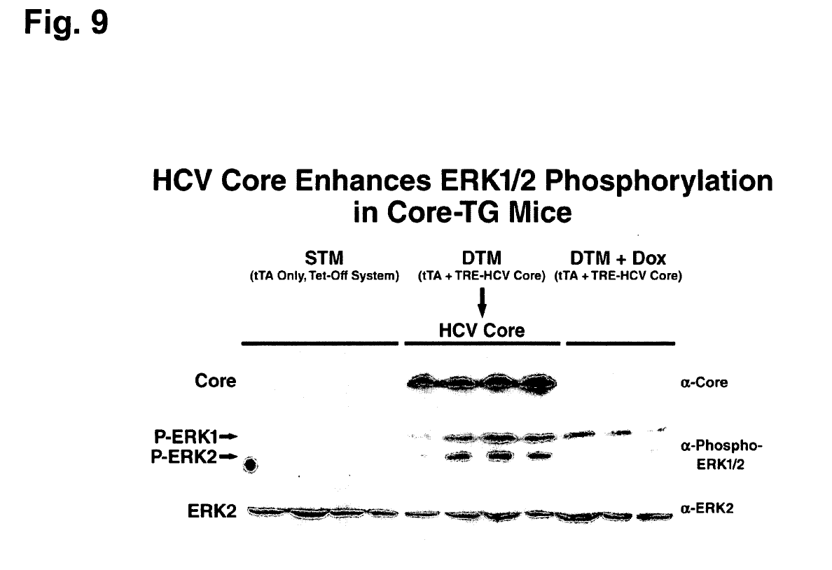

The results are shown in FIG. 9. As shown in FIG. 9, expression of HCV Core resulted in hyperphosphorylation of ERK2.

### Example 5

### MEK1/2 Inhibition does not Impair Viral Entry

Virus: Transfection of Huh7.5 cells with eGFP-Jc1 reporter construct RNA via electroporation (10 μg RNA per 4×10⁶ cells)

Harvest virus in the supernatant (Filter & Concentrate using Amicon MWCO 100): conc 10 ml to 200 μl after 3 days

Day 0: Plate Huh7.5 at 1×10⁴ cells per well at day 2 (24 well plate)

Day 1: Pretreat cells with 10 μl DMSO or 10 μM U0126 for 1 h

Infection with 50 μl conc. virus per well for 3 h at 37° C.

Addition of 10 μg/ml anti-CD81 antibody (α-CD81) for single-round infection

Day 4: Harvest infected cells for FACS analysis.

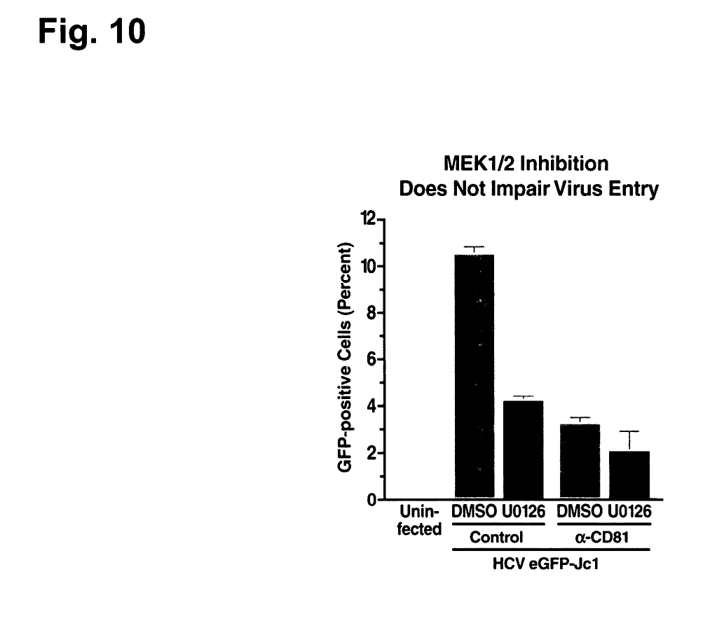

The results are shown in FIG. 10. In the presence of the MEK inhibitor U0126, HCV entered the cells, as detected by GFP expression.

### Example 6

### MEK1/2 Inhibition Blocks Viral Assembly

Day 0: Transfection of Huh7.5 cells with eGFP-Jc1 reporter construct RNA via electroporation (10 g RNA per 4×10⁶ cells)

Plate 2×10⁶ cells per T75 flask

Day 1: Wash cells

Addition of 10 μl DMSO or 10 μM U0126

Day 3: Harvest intracellular infectious particles by 3 freeze thaw cycles following centrifugation to remove cell debris.

Infection:

Huh7.5 plated at 5×10⁴ cells per well at day 2 (24 well plate)

Infection with 50 μl virus per well for 3 h at 37° C.

Wash 3×

Day 5: Harvest infected cells for FACS analysis

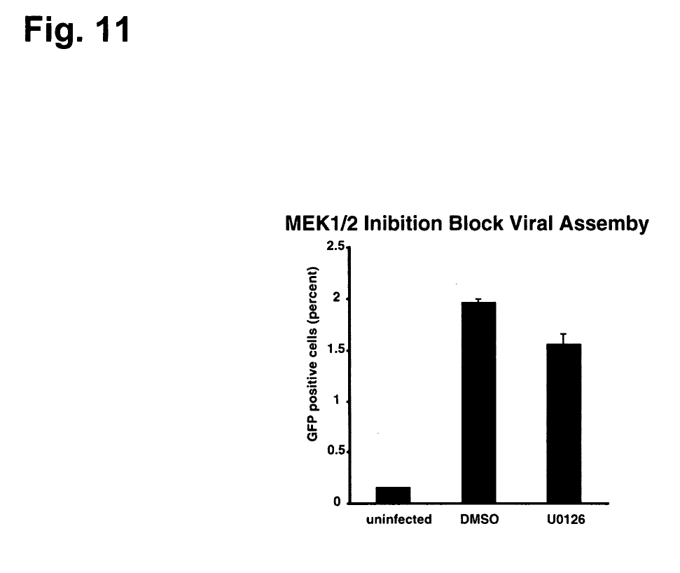

The results are shown in FIG. 11, and show that MEK1/2 inhibition blocks viral assembly.

### Example 7

### MEK1/2 Inhibition Enhances HCV RNA Replication

Day 0: Transfection of Huh7.5 cells with eGFP-Jc1 reporter construct RNA via electroporation (10 μg RNA per 4×10⁶ cells)

Plate 2×10⁶ cells per T75 flask

Day 1: Wash cells

Addition of 10 μl DMSO, 10 μM FPT inhibitor III, 50 μM PD98059, 10 μM ZM33672, 10 μM SB203580.

Day 3: Total cellular RNA was isolated using RNA Stat reagent (TelTest) according to the manufacturer's protocol and treated with the TURBO DNA-Free™ DNAse (Ambion). cDNA was synthesized using Superscript III reverse transcriptase (Invitrogen) with random hexamer primers, followed by RNase H (NEB) digestion. For qPCR we used HCV-specific primers and Taqman probe (Applied Biosystems) described previously (sense: 5′-CGGGAGAGCCATAGTGG-3′, antisense: 5′-AGTACCACAAGGCCTTTCG-3′, probe: 5′-CTGCGGAACCGGTGAGTACAC-3′) and pre-designed 18S rRNA Taqman assays (Applied Biosystems). Real-time PCR was performed using QuantiTect Probe PCR Kit (Qiagen) on a 7900HT Fast Real-time RT-PCR System (Applied Biosystems).

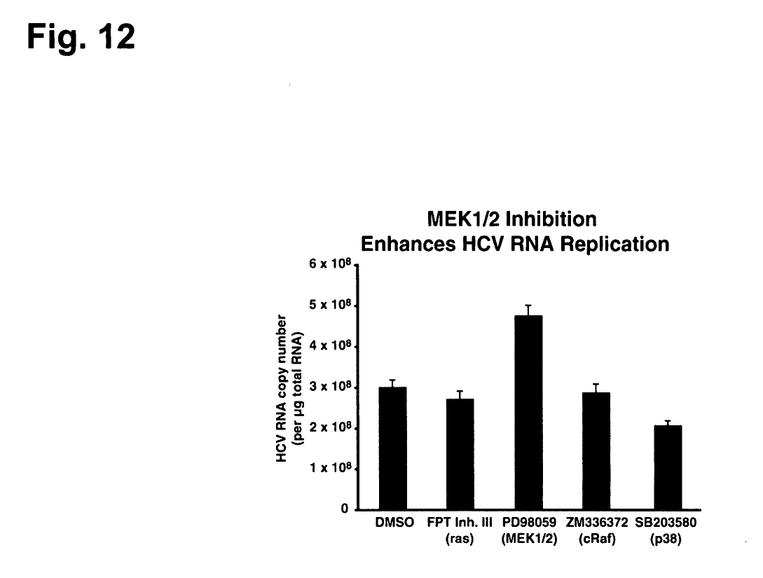

The results, presented in FIG. 12, show that MEK1/2 inhibition enhances HCV RNA replication

### Example 8

### MAPK Inhibitors Block HCV RNA Release

Day 0: Transfection of Huh7.5 cells with eGFP-Jc1 reporter construct RNA via electroporation (10 μg RNA per 4×10⁶ cells)

Plate 2×10⁶ cells per T75 flask

Day 1: Wash cells

Addition of 10 μl DMSO, 10 μM FPT inhibitor III (Ras inhibitor), 50 μM PD98059 (MEK1/2 inhibitor), 10 μM ZM33672 (cRaf inhibitor), 10 μM SB203580 (p38 MAPK inhibitor).

Day 2, 3, 4: Viral RNA from the culture supernatant was isolated with the MagMAX™ Viral RNA Isolation Kit (Ambion). RNA levels were adjusted to carrier RNA input that was added in excess prior RNA isolation. cDNA was synthesized using Superscript III reverse transcriptase (Invitrogen) with random hexamer primers, followed by RNase H (NEB) digestion. For qPCR HCV-specific primers and Taqman probe (Applied Biosystems) ((sense: 5′-CGGGAGAGCCATAGTGG-3′ (SEQ ID NO:16), antisense: 5′-AGTACCACAAGGCCTTTCG-3′ (SEQ ID NO:17), probe: 5′-CTGCGGAACCGGTGAGTACAC-3′ (SEQ ID NO:18)), and pre-designed 18S rRNA Taqman assays (Applied Biosystems) were used. Real-time PCR was performed using QuantiTect Probe PCR Kit (Qiagen) on a 7900HT Fast Real-time RT-PCR System (Applied Biosystems).

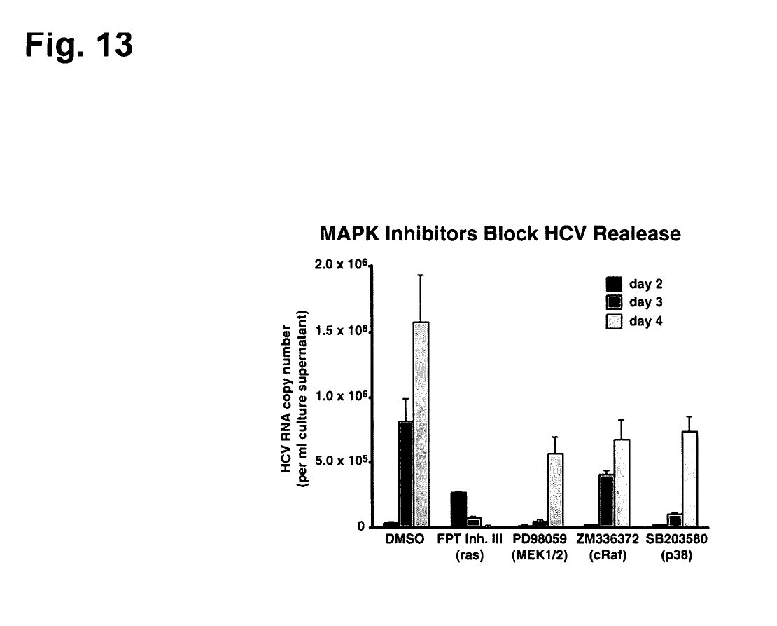

The results, shown in FIG. 13, show that MAPK inhibitors block HCV RNA release.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a hepatitis C virus (HCV) infection in an individual, the method comprising administering to the individual an effective amount of an agent that inhibits a RAS-RAF-MEK-ERK pathway.

2. The method of claim 1, wherein the agent inhibits an activity of a Ras polypeptide.

3. The method of claim 1, wherein the agent inhibits an activity of a Raf polypeptide.

4. The method of claim 1, wherein the agent inhibits an activity of a Mek polypeptide.

5. The method of claim 1, wherein the agent inhibits an activity of an Erk polypeptide.

6. The method of claim 1, further comprising administering to the individual an effective amount of at least a second therapeutic agent that treats an HCV infection.

7. The method of claim 6, wherein the at least a second therapeutic agent is an NS5B RNA-dependent RNA polymerase inhibitor.

8. The method of claim 6, wherein the at least a second therapeutic agent is an NS3 inhibitor.

9. The method of claim 6, wherein the at least a second therapeutic agent is a nucleoside analog.

10. The method of claim 9, wherein the nucleoside analog ribavirin, levovirin, viramidine, an L-nucleoside, or isatoribine.

11. The method of claim 6, wherein the at least a second therapeutic agent is an interferon-alpha (IFN-α).

12. The method of claim 11, wherein the IFN-α is pegylated IFN-α.

13. The method of claim 11, wherein the IFN-α is consensus IFN-α.

14. The method of claim 1, wherein the HCV-infected individual is a treatment-naïve individual.

15. The method of claim 1, wherein the HCV-infected individual failed a prior treatment for HCV infection.

16. The method of claim 1, wherein the HCV is HCV genotype 1.
